(19) United States
(12) Patent Application Publication
(10) Pub. No.: US 2007/0282221 A1
(43) Pub. Date: Dec. 6, 2007

(54) ULTRASOUND ASSISTED AND X-RAY ASSISTED BIOPSY DEVICES

(75) Inventors: Shih-Ping WANG, Los Altos, CA (US); Tor C. Anderson, Mountain View, CA (US)

Correspondence Address: BRIAN J. DAIUTO, 886 ILIMA COURT, PALO ALTO, CA, 94306, US

(73) Assignee: U-SYSTEMS, INC., San Jose, CA (US)

(21) Appl. No.: 11/757,996

(22) Filed: Jun. 4, 2007

Related U.S. Application Data
(60) Provisional application No. 60/803,762, filed on Jun. 2, 2006.

(51) Int. Cl. A61B 10/00

(52) U.S. Cl. 600/564

(57) ABSTRACT

Improved systems and related methods for x-ray assisted breast biopsy and ultrasound-assisted breast biopsy are described. In one preferred embodiment, for example, an apparatus for facilitating both x-ray assisted and ultrasound assisted breast biopsy is provided, comprising a biopsy table for supporting a patient in a prone position between first and second opposing ends thereof, the biopsy table having first and second openings, respectively, toward each of the first and second ends, through which the patient's breast may project depending on the patient's orientation on the biopsy table. The apparatus further comprises an ultrasound-assisted biopsy system positioned beneath the first opening, and an x-ray-assisted biopsy system positioned beneath the second opening. In other preferred embodiments, the ultrasound-assisted biopsy system and x-ray assisted biopsy system are mechanically interchangeable beneath a single opening. In other preferred embodiments, novel integrations between specified components of the ultrasound-assisted and x-ray-assisted biopsy systems are provided.

### CROSS-REFERENCE TO RELATED APPLICATIONS

This patent application claims the benefit of U.S. Ser. No. 60/803,762, filed Jun. 2, 2006. The subject matter of this provisional patent specification generally relates to the subject matter of the following commonly assigned disclosures, each of which is incorporated by reference herein: U.S. Ser. No. 60/746,259 filed May 2, 2006; US 2003/007598A1 published Jan. 9, 2003; WO 2004/030523A2 published Apr. 15, 2004; U.S. Ser. No. 60/565,698 filed Apr. 26, 2004; U.S. Ser. No. 60/577,078 filed Jun. 4, 2004; U.S. Ser. No. 60/629,007 filed Nov. 17, 2004; U.S. Ser. No. 60/702,202 filed Jul. 25, 2005; U.S. Ser. No. 60/713,322 filed Aug. 31, 2005; WO 2005/104729A2 published Nov. 10, 2005; and WO 2005/120357A1 published Dec. 22, 2005.

### FIELD

This provisional patent specification relates to image-guided biopsy procedures. More particularly, this provisional patent specification relates to devices for ultrasound assisted and x-ray assisted biopsy.

### BACKGROUND

Biopsy refers generally to the removal of a tissue sample from a living body for examination. In the field of breast cancer detection and treatment, breast tissue biopsies are often required when a suspicious lesion has been detected. Alternatives to traditional open surgical biopsy have been developed that are less invasive and, therefore, less risky and less costly. Percutaneous breast biopsy refers to the use of a biopsy needle or other instrument, usually long and relatively narrow, to puncture through the skin and capture cellular tissue associated with a breast lesion. The captured tissue is removed from the body and examined for a determination of whether the breast lesion represents a benign or malignant condition.

Percutaneous breast biopsy procedures include fine needle aspiration, core needle biopsy, and vacuum-assisted biopsy. In fine needle aspiration, a fine gauge needle (22 or 25 gauge) and a syringe are used to sample fluid from a breast cyst or remove clusters of cells from a solid mass. In core needle biopsy, small samples of tissue are removed using a hollow “core” needle. In vacuum-assisted biopsy, a special biopsy probe is inserted through a small opening in the skin. Unlike core needle biopsy, which requires several separate needle insertions to acquire multiple samples, the special biopsy probe used during vacuum-assisted biopsy is inserted only once for obtaining multiple samples. Vacuum-assisted biopsy is often referenced by the brand name of the biopsy instrument used, such as MAMMOTOME® from Johnson & Johnson Ethicon Endo-Surgery, MIBB® (Minimally Invasive Breast Biopsy) from Tyco International, and Intact™ Breast Lesion Excision System from Intact Medical Systems.

Percutaneous breast biopsy procedures are usually performed under image guidance using either stereotactic mammography or ultrasound, with the patient in either the upright or prone position. Another type of image-guided breast biopsy procedure is large core biopsy, also referenced by the brand name ABBI®, wherein entire lesions ranging in size from 5 mm to 20 mm can be removed. Although more invasive than the above-described fine needle aspiration, core needle biopsy, and vacuum-assisted biopsy procedures, ABBI® is still less invasive than traditional open surgical biopsy. According to Imaginis, an online breast cancer resource, large core biopsy requires the use of a prone biopsy table, in which the patient lies face-down and the breast extends downward through a hole on the table, whereas fine needle aspiration, core needle biopsy, and vacuum-assisted biopsy can be performed with the patient in an upright position or a prone position.

It is to be appreciated that the various preferred embodiments described infra can be applicable for the above-described biopsy procedures and any other procedure in which a biopsy instrument is directed to a lesion, either automatically or manually, under x-ray and/or ultrasound image guidance. Moreover, although described infra in the context of breast biopsy, it is to be appreciated that the scope of the present teachings extends to a variety of different medical or veterinary contexts in which ultrasound and/or x-ray assisted biopsy procedures are required, as would be apparent to one skilled in the art in view of the present disclosure.

Stereotactic mammography is known in the art and described, for example, in U.S. Pat. No. 5,078,142 (Siczek, et. al.) for prone positioning, and in U.S. Pat. No. 5,213,100 (Summ) for upright positioning, each of these references being incorporated by reference herein. By acquiring x-ray mammograms at two different angles of incidence, such as plus 15 degrees and minus 15 degrees, stereotactic mammography can yield exact location of the lesion for guidance of the biopsy instrument thereto.

For ultrasound assisted biopsy, the radiologist or surgeon will watch the biopsy instrument in real-time on the ultrasound monitor to help guide it to the lesion. In such applications, it is necessary to keep the biopsy needle positioned within the imaged plane in order for it to remain visible on the ultrasound monitor during the procedure. As used herein, the terms radiologist, physician, surgeon, etc. are used interchangeably and generically to refer to medical professionals that analyze medical images and make clinical determinations therefrom, and/or that perform medical procedures under the at least partial guidance of medical imaging systems, it being understood that such person might be titled differently, or might have differing qualifications, depending on the country or locality of their particular medical environment.

Percutaneous ultrasound guided biopsy of the breast is a procedure that can be quickly performed free-handed by a “skilled” physician, using a hand-held ultrasound imaging system, in an out-patient environment. Because this procedure would take much less physician time, it is much less expensive than other breast biopsy procedures, such as x-ray guided stereotactic biopsy and surgical biopsy. Thus, percutaneous ultrasound guided biopsy has become a highly popular breast biopsy procedure. However, ultrasound guided biopsy procedure could become even more popular if it were easier to perform. This is because many physicians are not coordinated enough to do the free-handed procedure, which requires the physician to hold a hand-held ultrasound transducer in one hand and the biopsy needle in the other hand, while looking at both the display monitor and the patient breast (usually placed three feet apart) and trying visualize simultaneously the thin biopsy needle (approximately 1 mm in diameter) and the breast lesion in the thin (approximately 1 mm thick) scan plane of the ultrasound imaging system.

U.S. Pat. No. 6,459,925 (Nields, et. al.), which is incorporated by reference herein, discusses a prone breast biopsy apparatus providing x-ray assisted biopsy and ultrasound assisted biopsy. However, one or more shortcomings arises that are addressed by one or more of the preferred embodiments herein. It would be desirable to facilitate a breast biopsy procedure in a manner that improves at least one of sample quality, thoroughness, patient comfort, image quality, and overall quickness of the process. It would be further desirable to provide for differing types and combinations of x-ray guidance and ultrasound guidance for biopsy procedures in a manner that promotes one or more of increased time efficiency, spatial efficiency (e.g., floor space), and functional flexibility. Other issues arise as would be readily apparent to one skilled in the art in view of the present disclosure.

### SUMMARY

Improved systems and related methods for x-ray assisted breast biopsy and ultrasound-assisted breast biopsy are provided. In one preferred embodiment, for example, an apparatus for facilitating both x-ray assisted and ultrasound assisted breast biopsy is provided, comprising a biopsy table for supporting a patient in a prone position between first and second opposing ends thereof, the biopsy table having first and second openings, respectively, toward each of the first and second ends, through which the patient's breast may project depending as a function of the patient's orientation on the biopsy table. The apparatus further comprises an ultrasound-assisted biopsy system positioned beneath the first opening, and an x-ray-assisted biopsy system positioned beneath the second opening. In other preferred embodiments, the ultrasound-assisted biopsy system and x-ray assisted biopsy system are mechanically interchangeable beneath a single opening. In other preferred embodiments, novel integrations between specified components of the ultrasound-assisted and x-ray-assisted biopsy systems are provided.

### BRIEF DESCRIPTION OF THE DRAWINGS

FIG. 1A illustrates a front view of an ultrasound-assisted and x-ray assisted biopsy device according to a preferred embodiment, including a prone patient positioned in an ultrasound assisted biopsy orientation;

FIG. 1B illustrates a front view of the ultrasound-assisted and x-ray assisted biopsy device of FIG. 1A including a prone patient positioned in an x-ray assisted biopsy orientation;

FIG. 1C illustrates a bottom view of an ultrasound-assisted and x-ray assisted biopsy device according to a preferred embodiment in an x-ray assisted biopsy configuration;

FIG. 1D illustrates a bottom view of the ultrasound-assisted and x-ray assisted biopsy device of FIG. 1C as it is pivotably interchanged between an x-ray assisted biopsy configuration and an ultrasound-assisted biopsy configuration;

FIG. 1E illustrates a bottom view of the ultrasound-assisted and x-ray assisted biopsy device of FIGS. 1C-1D in an ultrasound-assisted biopsy configuration;

FIG. 1F illustrates a bottom view of an ultrasound-assisted and x-ray assisted biopsy device according to a preferred embodiment in an x-ray assisted biopsy configuration;

FIG. 1G illustrates a bottom view of the ultrasound-assisted and x-ray assisted biopsy device of FIG. 1F as it is translationally interchanged between an x-ray assisted biopsy configuration and an ultrasound-assisted biopsy configuration;

FIG. 1H illustrates a bottom view of the ultrasound-assisted and x-ray assisted biopsy device of FIGS. 1F-1G in an ultrasound-assisted biopsy configuration;

FIG. 1I illustrates a bottom view of an ultrasound-assisted and x-ray assisted biopsy device according to a preferred embodiment in an x-ray assisted biopsy configuration;

FIG. 1J illustrates a bottom view of the ultrasound-assisted and x-ray assisted biopsy device of FIG. 1I after it has been translationally interchanged to an ultrasound-assisted biopsy configuration;

FIG. 1K illustrates a perspective view of a compressive member of an ultrasound-assisted biopsy system of an ultrasound-assisted and x-ray assisted biopsy device according to a preferred embodiment;

FIG. 2A illustrates a perspective view of an ultrasound-assisted and x-ray assisted biopsy device according to a preferred embodiment in an x-ray assisted biopsy configuration;

FIG. 2B illustrates a perspective view the ultrasound-assisted and x-ray assisted biopsy device of FIG. 2A after it has been slidably switched to an ultrasound assisted biopsy configuration;

FIG. 2C illustrates a perspective view of an ultrasound-assisted and x-ray assisted biopsy device according to a preferred embodiment as it is being slidably switched between x-ray assisted and ultrasound-assisted biopsy configurations;

FIG. 2D illustrates a perspective exploded view of a dual-modality compressive member including an ultrasound probe and a scanning x-ray detector according to a preferred embodiment;

FIG. 2E illustrates a perspective view of an ultrasound-assisted and x-ray assisted biopsy device according to a preferred embodiment;

FIG. 3 illustrates a method and associated apparatus for facilitating ultrasound-assisted biopsy according to a preferred embodiment;

FIG. 4 illustrates a perspective exploded view of a compressive member including a real-time 3D scanning probe according to a preferred embodiment; and

FIG. 5 illustrates a perspective exploded view of a compressive member including a large multiple-mode linear array ultrasound scanning probe according to a preferred embodiment.

### DETAILED DESCRIPTION

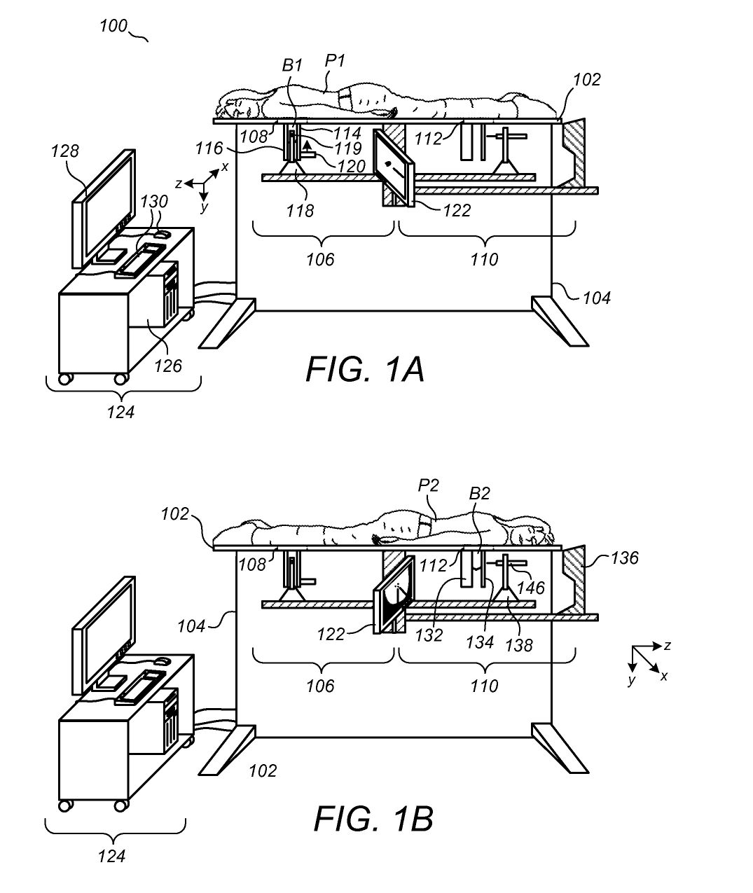

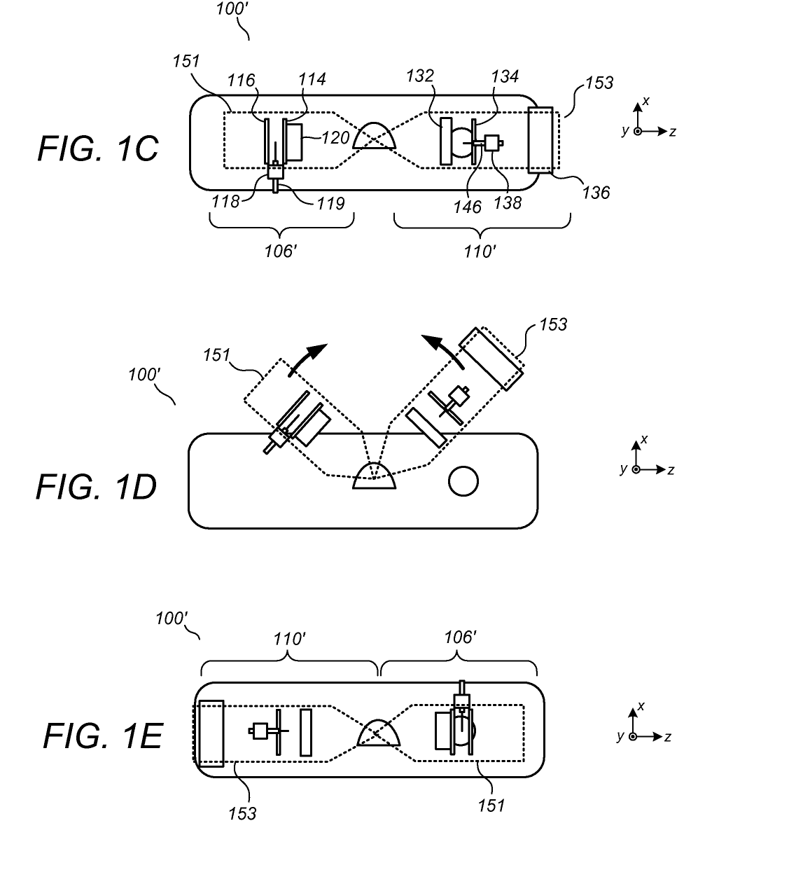

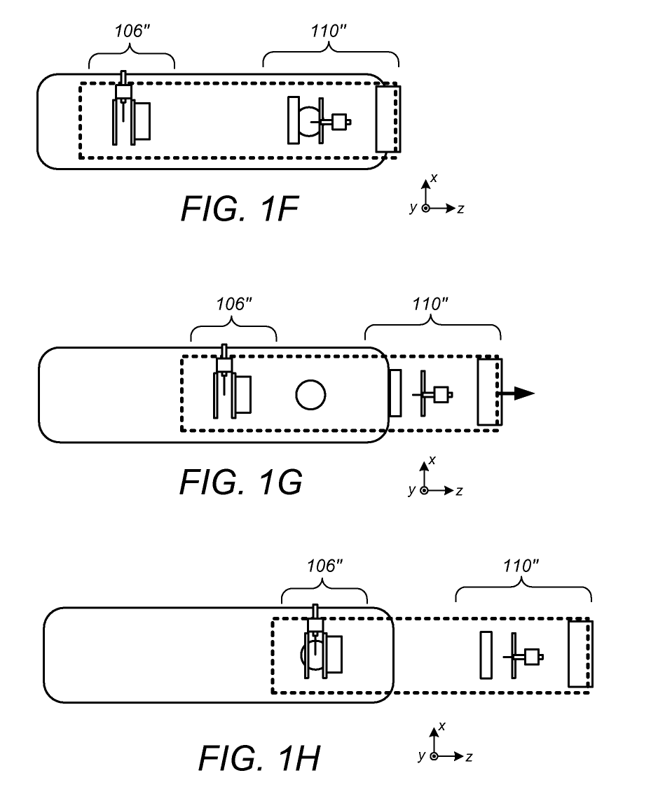

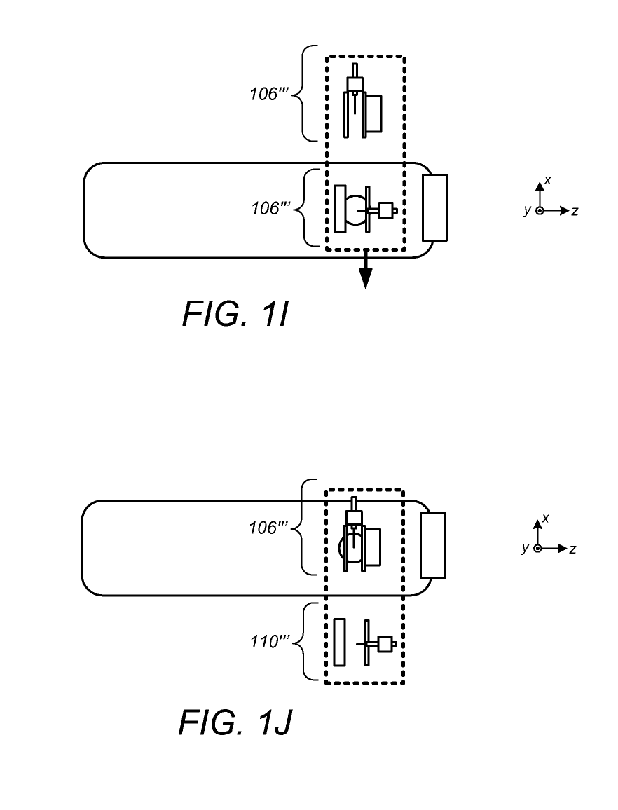

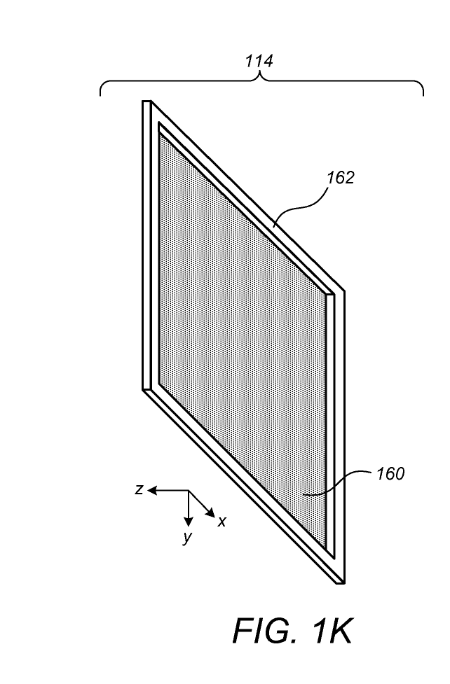

With reference to the preferred embodiments of FIGS. 1A-1J, it has been found that there is often an “either-or” decision between x-ray assisted biopsy and ultrasound assisted biopsy for a particular breast lesion. More specifically, x-ray assisted biopsy is generally better for microcalcifications because x-ray can see the microcalcifications better than ultrasound. Also, the stereotactic x-ray pictures are comparable to the x-ray mammograms that were used for the original screening/diagnosis so there is a desirable visual familiarity for the doctor. In contrast, ultrasound assisted biopsy is generally better for masses because the ultrasound images can see the mass better (not occluded by out-of-plane tissue). Also, the ultrasound images are comparable to what was seen in the hand-held ultrasound images during the diagnosis. It would be desirable to provide a system that can facilitate either procedure while also saving cost and/or floor space.

With reference to FIGS. 1A-1B, according to one preferred embodiment, an image-guided biopsy system 100 is provided that facilitates separate x-ray assisted biopsy and ultrasound assisted biopsy procedures while preserving valuable floor space in the clinic. More particularly, image-guided biopsy system 100 comprises an ultrasound assisted biopsy system 106 at a first end of a prone biopsy table 102 and an x-ray assisted biopsy system 110 at a second end. For a first patient P1 for which ultrasound assisted biopsy is required, the patient P1 lies with their breast B1 projecting through a first opening 108 at the first end for access by the ultrasound assisted biopsy system 106. For a second patent P2 for which x-ray assisted biopsy is required, the patient P2 lies with their breast B2 projecting through a second opening 112 at the second end for access by the x-ray assisted biopsy system 110.

Various components of the system 100 are shared between the ultrasound assisted system 106 and the x-ray assisted system 110 to preserve space and/or cost. For example, a common control station 124 comprising a processor 126, a display 128, and input devices 130 is used for administration, data access/archiving, image processing, computer-aided diagnosis, servo-motor control for the various moveable elements, and so on, for both the ultrasound assisted and x-ray assisted biopsy procedures. As another example, a real-time medical image display monitor 122 is provided that is movably mounted so that it can be used for both the ultrasound assisted and x-ray assisted biopsy procedures.

Ultrasound assisted biopsy system 106 comprises a first compressive member 114 for contacting a lower (inferior) side of the breast and a second compressive member 116 for contacting an upper (superior) side of the breast. An ultrasound probe 120 is swept across the first compressive member 114 to ultrasonically scan the breast therethrough, and a three-dimensional volumetric representation of the breast is digitally formed from the ultrasonic scans. A biopsy guide 118 guides a biopsy instrument 119 to and from the lesion in the breast. Each of the first compressive member 114, second compressive member 116, biopsy guide 118, and ultrasound probe 120 is coupled to servo-motors or other actuation devices (not shown) to achieve the movement functionalities described herein.

The first compressive member 114, second compressive member 116, biopsy guide 118, and ultrasound probe 120 are further mounted so as to be rotatable in the aggregate around the y-axis (i.e., in a sagittal plane around an anterior-posterior axis) so that the compression can be in the craniocaudal direction, lateral direction, or at any mediolateral angle therebetween. For compression in the lateral direction, the first compressive member 114 compresses the medial (inner) side of the breast while the second compressive member 116 compresses the outer side of the breast. As used herein, for preferred embodiments in which the direction of compression is so rotationally adjustable, whether in the context of ultrasound assisted biopsy, x-ray assisted biopsy, or combined ultrasound and x-ray assisted biopsy, “lower/inner” and “upper/outer” will refer to the sides of the breast that are being compressed by the first and second compressive members, respectively. This terminology, however, is not to be construed as limiting the scope of the preferred embodiments. More particularly, this terminology is not to be construed as limiting the scope of the preferred embodiments to counterclockwise (FIG. 1A) rotation of the assembly relative to the patient (clockwise for FIG. 1B). Rather, the scope of the preferred embodiments also encompasses the converse scenario in which the assembly is rotatable in the clockwise direction (FIG. 1A, counterclockwise for FIG. 1B) and for which the first and second compressive members are for the lower/outer and upper/inner surfaces, respectively.

For one preferred embodiment, the ultrasound probe 120 and biopsy guide 118 are mechanically connected such that the scan plane is kept coplanar with the biopsy instrument 119, in a manner similar to that described in U.S. 60/746,259, supra, wherein the mechanical connection is also releasable. Subsequent to stabilization of the breast and prior to insertion of the biopsy instrument 119, the ultrasound probe 120 and biopsy guide 118 are decoupled, and the ultrasound probe 120 is swept across the entire breast to acquire a complete volumetric representation of the breast, including the lesion to be biopsied. The physician can view various 2D/3D views of the breast volume to plan for the insertion point and trajectory of the biopsy instrument 119. The ultrasound probe 120 and biopsy guide 118 are then recoupled such that the scan plane is kept coplanar with the biopsy instrument 119, and remain so coupled during the insertion(s) and removal(s) of the biopsy instrument 119 from the lesion. Afterward, the ultrasound probe 120 and biopsy guide 118 are again decoupled for subsequent volumetric sweeps that allow the physician to closely examine the effect that the process has had on the lesion and the surrounding tissue.

X-ray assisted biopsy system 110 comprises a first compressive member 132 and a second compressive member 134 that compress a lower/inner surface and upper/outer surface of a breast B2 of a patient P2. The breast B2 projects downwardly through the opening 112. A biopsy guide 138 provides for the controlled insertion and removal of a biopsy instrument 146. The first compressive member 132 comprises an x-ray film cassette, an x-ray CCD detector, an x-ray computed radiography plate, or other x-ray recording device for receiving x-ray radiation emitted by an x-ray source 136 that is pivotably mounted for stereotactic imaging in the plane of the lesion of interest. Display 122 provides a computer-driven stereotactic imaging display or, alternatively, can serve as a lightbox for film images.

One particular advantage of the preferred embodiment of FIGS. 1A-1B over U.S. Pat. No. 6,459,925, supra, and over one or more of the other preferred embodiments herein, is that the functionality of the ultrasound assisted biopsy end is substantially unfettered and uncompromised by the presence of the x-ray assisted biopsy equipment, and vice versa, yet at the same time there is a savings in floor space and system cost. Each modality can be customized for optimal performance without requiring compromises associated with functional integration with the other modality.

FIGS. 1C-1E illustrate bottom views (looking upward away from the floor) of an image-guided biopsy system 100′ according to a preferred embodiment that is similar to that of FIGS. 1A-1B except that the ultrasound assisted biopsy system 106′ and x-ray assisted biopsy system 110′ are pivotably mounted to be interchanged with each other at a same end of the table (the right side in FIGS. 1C-1E), using pivot mountings 151 and 153 or other equivalent means. In still other preferred embodiments, image-guided biopsy systems are provided that are similar to that of FIGS. 1A-1B except that the ultrasound assisted biopsy system and x-ray assisted subsystem can be translationally interchanged along the length of the biopsy table, i.e., the z direction (FIGS. 1F-1H) and/or along the width of the biopsy table (FIGS. 1I-1J). Each of these configurations brings about one or more advantages, and equivalent setups having similar physical interchangeability of the ultrasound assisted biopsy system and the x-ray assisted biopsy system are within the scope of the preferred embodiments. For yet another preferred embodiment (not shown), an existing x-ray assisted biopsy table is retrofitted to be a combined x-ray assisted biopsy and ultrasound assisted biopsy table, i.e., the above-described functionalities are provided using add-on components, which saves both floor space and upgrading cost.

With reference now to FIG. 1K, according to other preferred embodiments for use in conjunction with each of the above preferred embodiments and those hereinbelow, the compressive member through which the ultrasound probe scans the breast, such as the compressive member 114 of FIG. 1A, comprises a taut membranous sheet 160 stretched or otherwise disposed across a frame 162, as illustrated in FIG. 1K. The taut membranous sheet preferably comprises a vented membrane material as described in U.S. 60/702,202, supra, and/or a fabric material as described in U.S. 60/713,322, supra, the vented membrane or fabric material being substantially porous to acoustic couplant liquid or gel.

One or more important advantages are realized by using a vented membrane or taut fabric sheet that is substantially porous to acoustic couplant liquid according to the present teachings. For example, the vented membrane or taut fabric sheet promotes dissipation of air bubbles that might otherwise form in the acoustic couplant at the membrane surface. As compared to using a material nonporous to the acoustic couplant, image quality is increased by virtue of fewer air bubbles being present between the ultrasound transducer and the tissue surface. As another example, in embodiments for which the vented membrane or taut fabric sheet is sheer (i.e., thin, fine, and relatively transparent), easier viewing of the breast is provided therethrough (as compared to using a relatively opaque material), which further facilitates positioning of the breast and monitoring of the scanning process. As yet another example, as will be discussed further infra for other preferred embodiments in which the biopsy instrument is perpendicular to the plane of compression, the vented membrane or taut fabric sheet can be readily designed to allow puncture therethrough by a biopsy instrument while still remaining intact, thereby allowing more flexibility in placement of the biopsy instrument. Another advantage of using a taut membranous sheet is that patient comfort is promoted by virtue of some amount of conformal “give” or bowing of the sheet when compressing the breast.

Preferably, the vented membrane or taut fabric sheet is wetted with an acoustic couplant facilitating acoustic coupling between the ultrasound transducer and the tissue sample. The vented membrane or taut fabric sheet may be wetted with the acoustic couplant by one or more of: (i) pre-impregnating the vented membrane or taut fabric sheet with the acoustic couplant; (ii) applying the coupling agent to a tissue-facing surface of the vented membrane or taut fabric sheet, or to the tissue surface, prior to compressing the tissue sample; (iii) applying the coupling agent to a transducer-facing surface of the vented membrane or taut fabric sheet prior to compressing the tissue sample; and (iv) applying the coupling agent to a transducer-facing surface of the vented membrane or taut fabric sheet subsequent to compressing the tissue sample and prior to the scanning.

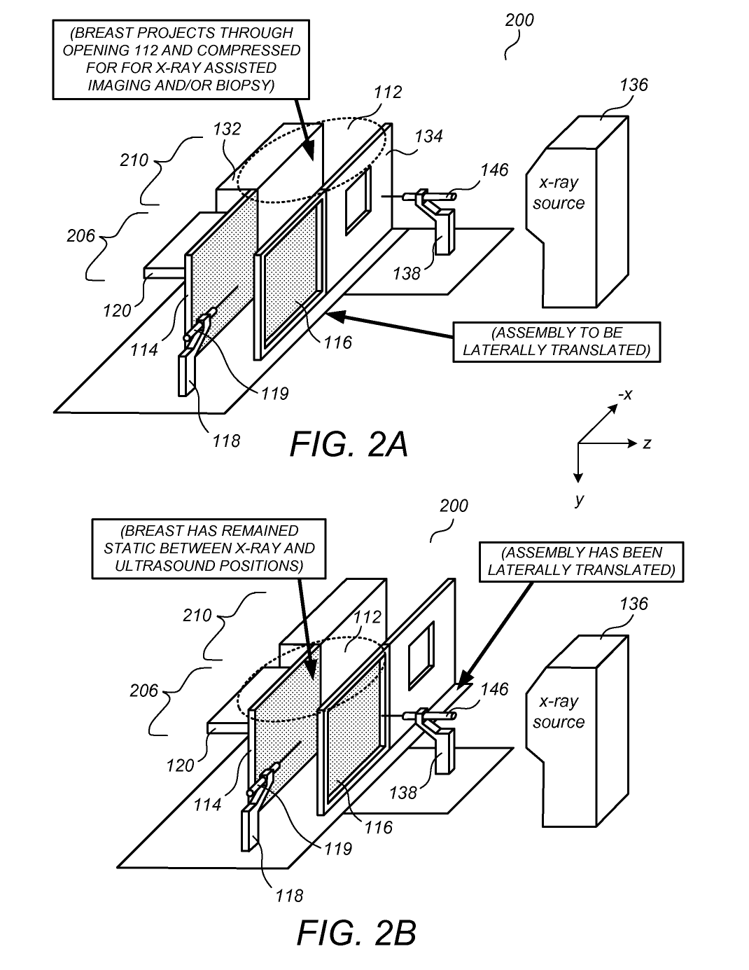

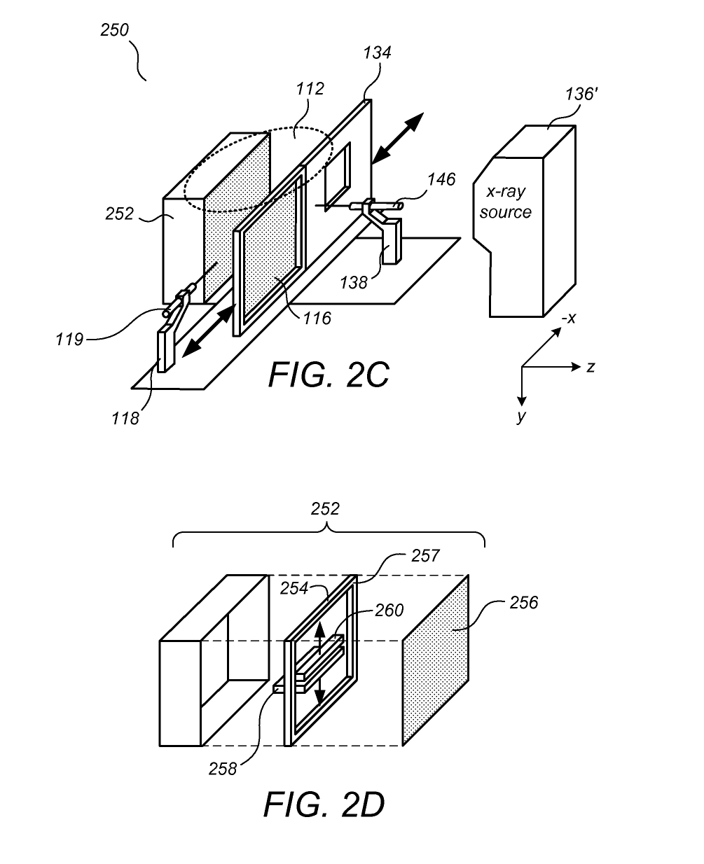

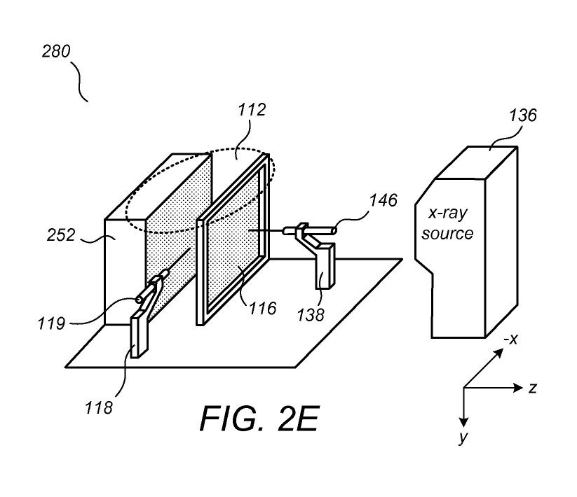

In other cases, it has been found desirable to allow for dual-modality assisted biopsy in which both ultrasound and x-ray guidance is provided. Accordingly, in other preferred embodiments, a higher degree of integration between the ultrasound assisted biopsy system and the x-ray assisted biopsy system is provided, as described further herein with reference to FIG. 2A-2B, providing one or more of increased cost savings, convenience, and real-time flexibility.

With reference to FIGS. 2A-2B, a prone breast biopsy system 200 is provided with several components similar to those of FIGS. 1A-1B except that there is now provided a slidable, real-time interchange between x-ray assisted biopsy and ultrasound assisted biopsy modalities. For clarity of description herein, various components such as the table and table frame are omitted and only the relevant subsystems are illustrated, as one skilled in the art would readily be able to mount or otherwise integrate the described components with the remainder of the overall system in view of the present disclosure.

FIG. 2A illustrates the biopsy system 200 in a first configuration for x-ray-assisted biopsy. Immediately adjacent to, and substantially coplanar with, the x-ray compressive members are the ultrasound compressive members, forming a compression assembly in which the respective compressive members are actuated in unison. Subsequent to compression for x-ray assisted biopsy, and being sure that the biopsy instrument 146 has been removed (or was never yet inserted), the assembly is laterally translated (i.e., in the -x direction) to the configuration of FIG. 2B. When the breast skin has been lubricated with ultrasonic gel to facilitate sliding, it has been found that the breast maintains substantially the same volumetric shape between the x-ray mode and the ultrasound mode. Advantageously, the resulting x-ray and ultrasound images are therefore in appreciable registration and can be used for fusion imaging or other applications.

In the preferred embodiment of FIGS. 2A-2B, the x-ray assisted biopsy mode uses a first compressive member 132 for the lower/inner side of the breast and a second compressive member 134 for the upper/outer side of the breast, while the ultrasound assisted biopsy mode uses a third compressive member 114 for the lower/inner side of the breast and a fourth compressive member 116 for the upper/outer side of the breast. The first compressive member 132 comprises an x-ray detector assembly housing a standard 2D x-ray detector, and the second compressive member 134 comprises a rigid plate having an opening to allow the biopsy instrument 146 to enter the breast. The third compressive member 114 and fourth compressive member 116 comprise taut membranous sheets, preferably of the type described in relation to FIG. 1K, supra.

Advantageously, for the ultrasound assisted mode, an option is provided for either or both of the ultrasound guides/instruments 118/119 and 138/146 to be used. In the case of ultrasound guide/instrument 118/119, the biopsy instrument 119 will show up brightly in the planar images and 3D views generated by the ultrasound probe 120 when kept coplanar therewith. In the case of ultrasound guide/instrument 138/146, it is preferable that lateral beamsteering is used by the ultrasound probe 120 (e.g., where the interrogating acoustic pulses are directed in the x-z plane at an angle of 30-45 degrees away from the z-direction), so that the biopsy instrument 146 will show up sufficiently in the planar images and 3D views. Also in the case of ultrasound guide/instrument 138/146, the membrane of the fourth compressive member 116 is locally punctured to allow access to the breast therethrough, or may already be provided with holes at various places.

Referring now to FIG. 2C, according to another preferred embodiment, a prone breast biopsy system 250 is provided with certain components similar to those of FIGS. 2A-2B, but with a dual-modality compressive member 252 that remains fixed while the x-ray compressive member 134 and the ultrasound compressive member 116 are slidably interchanged between modes. With reference to FIG. 2D, the dual-modality compressive member 252 comprises an ultrasound probe 258 that is swept over a taut membranous sheet 256 for scanning the breast, the taut membranous sheet being disposed across a frame 257 and comprising a taut vented membrane or taut fabric sheet as described supra. The dual-modality compressive member further comprises a movable scanning x-ray detector 260, comprising a linear or small-area rectangular CCD array, that can be mechanically coupled with the ultrasound probe 258 to have a common scanning trajectory or, alternatively, that can be decoupled from the ultrasound probe 258 to have a different scanning trajectories at different times. The x-ray source 136′ is modified to accommodate the functionality of the movable scanning x-ray detector 260, in a manner similar to that discussed in WO 2006/015296 A2 (Fischer Imaging Corporation), which is incorporated by reference herein.

Referring now to FIG. 2E, according to another preferred embodiment, a prone breast biopsy system 280 is provided with several components analogous to those of FIGS. 2C-2D except that no slidable interchange is required and the ultrasound compressive member 116 is used for both ultrasound assisted biopsy and x-ray assisted biopsy modes. In the x-ray mode, the taut membranous sheet of the compressive member 116 can be punctured by the biopsy instrument 146. Advantageously, the breast does not need to be moved between the x-ray and ultrasound imaging processes, and therefore the images are inherently in registration with each other. In one alternative preferred embodiment, the biopsy guides/biopsy instruments 118/119 and 138/146 are replaced by a single biopsy guide/biopsy instrument assembly that is pivotably mounted to swing in the x-z plane around a y-oriented axis that passes through the opening 112. Optionally, as with other preferred embodiments, the entire assembly can be rotated to change the plane of compression among craniocaudal, lateral, and various mediolateral angles.

In another alternative preferred embodiment, a single-modality, ultrasound-only assisted biopsy system is provided that is similar to that of FIG. 2E except that the x-ray components (i.e., the movable CCD array detector and the x-ray source) are omitted. As discussed supra, the ultrasound probe can use in-plane beamsteering and/or can be a real-time 3D (“4D”) ultrasound probe (see infra) for better imaging when the biopsy instrument is inserted at angles near-perpendicular to the plane of compression. Optionally, a second ultrasound probe (not shown) can be provided that scans across the compressive member 116 into the breast volume, such that ultrasound scans are acquired from both sides of the compressed breast. In still another alternative preferred embodiment, a single-modality, x-ray-only assisted biopsy system is provided that is similar to that of FIG. 2E except that the ultrasound probe is omitted.

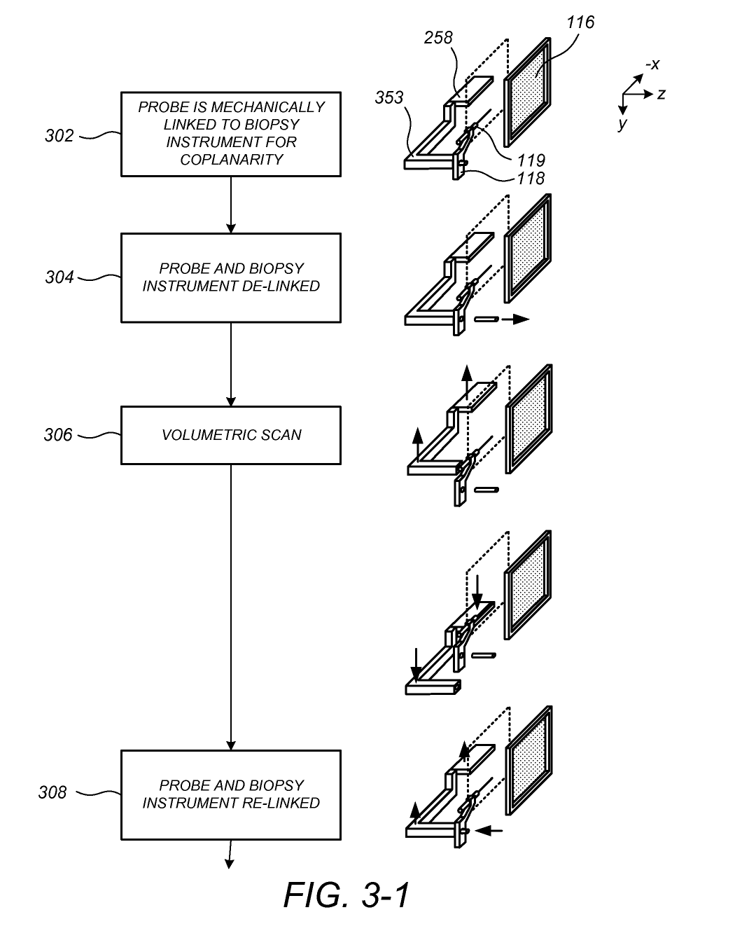

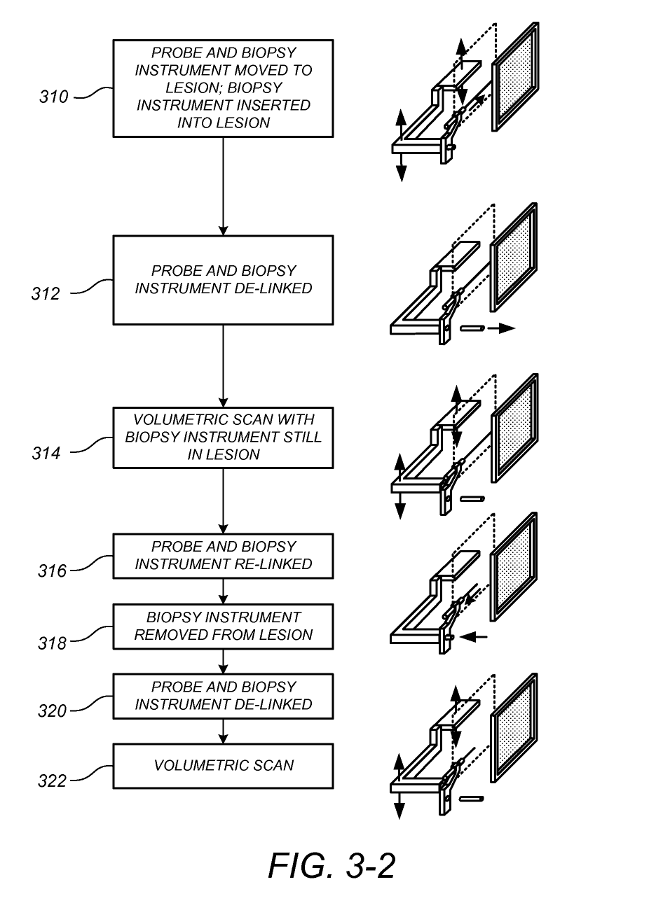

With reference now to FIG. 3, according to another preferred embodiment, an ultrasound-assisted biopsy device is provided having at least two modes of operation. In a first mode the ultrasound probe 258 is mechanically linked to the biopsy guide 118 in a manner that mechanically maintains the biopsy instrument 119 in the scan plane. In a second mode the ultrasound probe 258 is mechanically de-linked from the biopsy guide 118 to allow free volumetric scanning. Shown in FIG. 3 is a conceptual visualization of a releasable linkage mechanism 353 that achieves this functionality. The system can be switched between the first and second modes to allow for advantageous functionality.

For example, as illustrated in FIG. 3, starting from a linked configuration (step 302), the biopsy instrument is de-linked (step 304) to enter the de-linked mode. A preliminary “survey” or “exploratory” sweep is performed (step 306) that allows the physician to get a good overall view of the lesion and its surrounding context. The system then re-enters the linked mode (step 308), and the probe and biopsy instrument are moved in unison to the lesion location and the biopsy instrument is inserted (step 310). Next, the probe and biopsy instrument are again de-linked (step 312) and a volumetric scan performed (step 314) while the biopsy instrument is in the lesion, to allow the physician to get a good look at how well the lesion is being sampled. Removal of the biopsy instrument is then facilitated by re-linking the probe and the biopsy instrument (steps 316, 318). Finally, the effectiveness of the overall procedure can be assessed by again de-linking the probe and biopsy instrument (step 320, optional) and performing a volumetric scan (step 322). According to another preferred embodiment, the devices and methods of FIG. 3 are incorporated into a supine biopsy device comprising a scanning pod with biopsy instrument attachment as described in U.S. 60/746,259, supra.

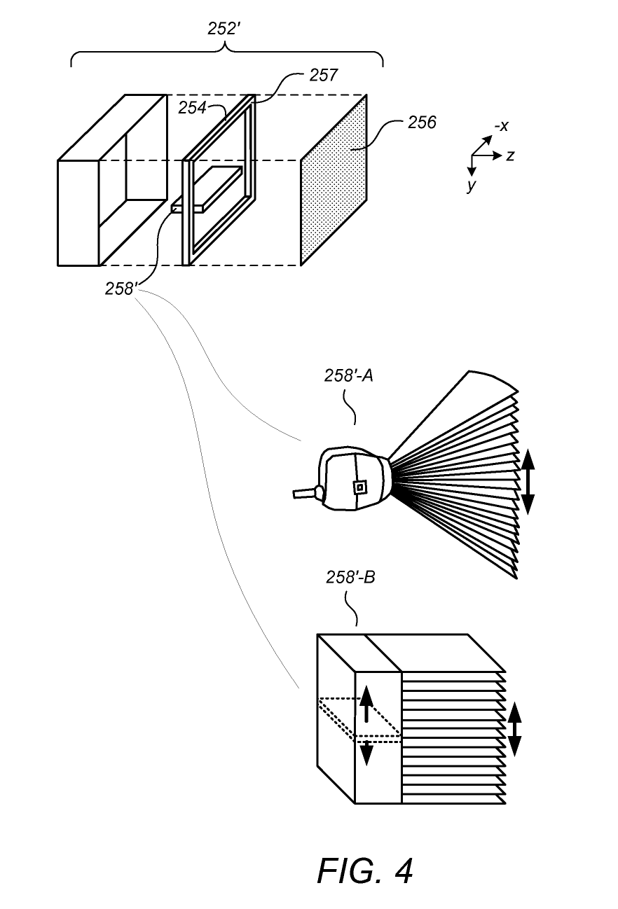

With reference to FIG. 4, in another preferred embodiment a single- or dual-modality assisted breast biopsy subsystem is provided including a compressive member 252′ that is similar to that of FIG. 2E, and wherein the ultrasonic probe 258′ comprises a real-time 3D scanning probe in which the probe is mechanically swept at a relatively high rate, e.g., an entire subvolume within its ambit is scanned at a rate of 0.5 times per second to 30 times per second. This allows the physician to view a real-time volumetric display (e.g., a maximum intensity projection image) or a real-time planar display for different planes within the ultrasound volume as the biopsy instrument is manipulated. One example of a suitable real-time 3D scanning probe is the VOLUSON 730 available from General Electric Medical Systems. Advantageously, by viewing of the real-time volumetric display, crucial real-time visual feedback is provided to the physician during manipulation of the biopsy instrument, even when the ultrasound probe is no longer “coplanar” with the biopsy instrument.

More generally, the real-time 3D scanning probe can be an angularly swept device such as 258′-A or a linearly swept device such as 258′-B. For one preferred embodiment, the linearly swept real-time 3D scanning probe 258′-B is relatively small compared to the overall compressive surfaces, using a 6 cm probe that is swiftly swept over a 6 cm×9 cm area. Preferably, the scanning surface (not shown) of the linearly swept real-time 3D scanning probe 258′-B device comprises the taut membranous sheet previously described. According to another preferred embodiment, the devices and methods of FIG. 4 are incorporated into a supine biopsy device comprising a scanning pod with biopsy instrument attachment as described in U.S. 60/746,259, supra.

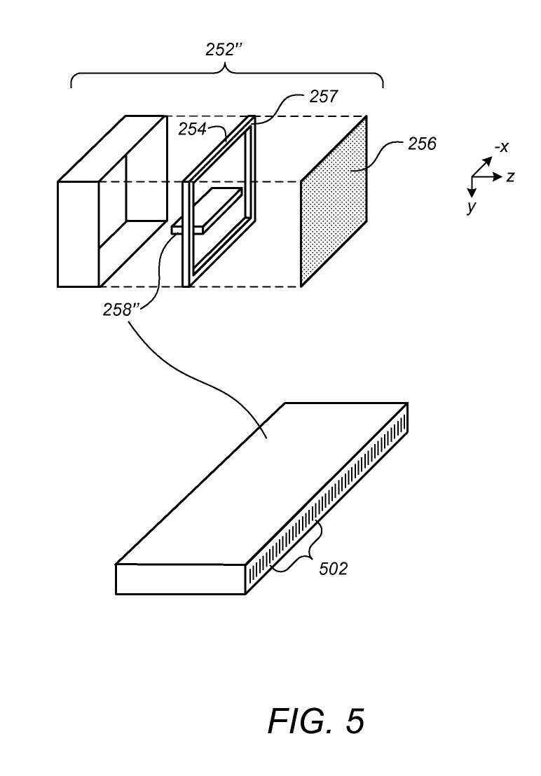

With reference to FIG. 5, in another preferred embodiment a single- or dual-modality assisted breast biopsy subsystem is provided including a compressive member 252″ that is similar to that of FIG. 2E, and wherein the ultrasonic probe 258″ therein comprises a relatively large transducer array, e.g., a 768-element linear array that is 15 cm long, having two modes of operation. In a first mode, most or all of the transducers along the length of the scanning probe are used to obtain a 3D image of the overall breast volume (or a “scout” or “exploratory” image), for which the scanning speed can be relatively slow, perhaps about 5-20 seconds to acquire the whole breast volume. As known in the art, it is usually the ultrasound computing hardware that limits the scanning speed rather than the mechanical aspects of the system. According to a preferred embodiment, in a second mode, most of the transducers are turned off and only a small portion 502 of the ultrasound probe is activated. Also, instead of sweeping in the y-direction across the entire distance of the compressive surface, a smaller distance such as 6 cm to 9 cm is traversed at the location of interest. Because a smaller number of transducer elements are used (e.g., 64 or 128 elements) and because of the smaller distance traversed, the ultrasound computing hardware can provide “live 3D” or “4D” images during the crucial steps of the biopsy procedure, similar to those provided by the real-time 3D or “4D” scanning probes of FIG. 4, supra, and at similar frame rates. Advantageously, a combination of the size advantages of a large probe and the speed advantages of a small probe are provided.

According to another preferred embodiment, the active aperture can “walk” across the probe 258″ via activation of successive sequences of transducers, thereby allowing a moving subvolume to be imaged in this fashion. For example, at a first time, the portion 502 would comprise transducers 1-64, at a second time the portion 502 would comprise transducers 5-68, at a third time the portion 502 would comprise transducers 9-72. Of course, included in this preferred embodiment would be a “jumping” aperture as well, allowing distal subvolumes to be imaged. For example, at a fourth time, the portion 502 would comprise transducers 1-64, and at a fifth time the portion 502 would comprise transducers 385-448.

Whereas many alterations and modifications of the present invention will no doubt become apparent to a person of ordinary skill in the art after having read the foregoing description, it is to be understood that the particular embodiments shown and described by way of illustration are in no way intended to be considered limiting. By way of example, although at least one preferred embodiment is described supra in the context of linear ultrasound transducers, it is to be appreciated that other transducer types including 1.25D, 1.5D, and 2D transducers can be used without departing from the scope of the preferred embodiments. By way of further example, although described primarily in terms of breast biopsy, one or more of the above-described preferred embodiments are readily applicable and/or adaptable for compressive biopsy for the arm, the leg, the neck, the abdomen, or other human or animal body part. Therefore, reference to the details of the embodiments are not intended to limit their scope.

1-3. (canceled)

4. An apparatus for facilitating image-guided breast biopsy procedures, comprising:
a biopsy table for supporting a patient in a prone position between first and second opposing ends of the biopsy table, the biopsy table having a first opening through which the prone patient's breast may project when the patient is in a first orientation relative to said first and second ends of the biopsy table, the biopsy table having a second opening through the prone patient's breast may project when the patient is in a second orientation relative to said first and second ends of the biopsy table;
an ultrasound-assisted biopsy system positioned beneath said first opening, the ultrasound-assisted biopsy system comprising an ultrasound probe, a first biopsy instrument insertion platform, and first and second compressive members for compressing the breast along a first plane generally perpendicular to said biopsy table;
an x-ray-assisted biopsy system positioned beneath said second opening, the x-ray-assisted biopsy system comprising an x-ray source, an x-ray detector, a second biopsy instrument insertion platform, and third and fourth compressive members for compressing the breast along a second plane generally perpendicular to said biopsy table; and
a display system coupled to each of said ultrasound-assisted biopsy system and said x-ray-assisted biopsy system for displaying medical image information generated thereby.

5. The apparatus of claim 4, said first biopsy instrument insertion platform comprising:
a first biopsy instrument for puncturing through a skin of the breast and capturing cellular tissue therein; and
a first biopsy guide supportably coupled to said first biopsy instrument for facilitating constrained linear movement of said first biopsy instrument within a plane substantially parallel to said first plane during said puncturing and capturing;
wherein said first biopsy instrument, said first biopsy guide, and said first and second compressive members are jointly rotatable around an axis generally perpendicular to said biopsy table such that said first plane is user adjustable.

6. The apparatus of claim 5, wherein said first plane is user adjustable to any of a craniocaudal (CC) direction, a mediolateral oblique (MLO) direction, and a lateral (LAT) direction.

7. The apparatus of claim 4, wherein said second orientation of the patient relative to said first and second ends of the biopsy table is substantially opposite said first orientation of the patient relative to said first and second ends of the biopsy table, and wherein said x-ray-assisted biopsy system comprises a stereotactic x-ray assisted biopsy system.

8. The apparatus of claim 4, said first compressive member comprising a first surface that contacts the breast during compression thereof and a second surface opposite said first surface, wherein said ultrasound-assisted biopsy system is configured such that said ultrasound probe is swept across said second surface to generate an ultrasound volume representative of the breast while compressed between said first and second compressive members.

9. The apparatus of claim 8, wherein said first compressive member comprises one of a vented membrane and a taut fabric sheet substantially porous to an acoustic couplant liquid or gel.

10. The apparatus of claim 9, said first biopsy instrument insertion platform comprising:
a first biopsy needle for puncturing through a skin of the breast and capturing cellular tissue therein during a biopsy procedure; and
a first biopsy guide supportably coupled to said first biopsy needle for facilitating constrained linear movement of said first biopsy instrument within a third plane during said biopsy procedure, said third plane being generally perpendicular to said first plane such that said first biopsy needle also punctures through said first compressive member during said biopsy procedure.

11. The apparatus of claim 9, said first biopsy instrument insertion platform comprising:
a first biopsy instrument for puncturing through a skin of the breast and capturing cellular tissue therein; and
a first biopsy guide supportably coupled to said first biopsy instrument for facilitating constrained linear movement of said first biopsy instrument within a plane substantially parallel to said first plane during said puncturing and capturing;
wherein said first biopsy instrument, said first biopsy guide, and said first and second compressive members are jointly rotatable around an axis generally perpendicular to said biopsy table such that said first plane is user adjustable.

12. The apparatus of claim 11, said first biopsy instrument being long and narrow in shape, said ultrasound probe being a linear ultrasound probe, wherein said linear ultrasound probe and first biopsy instrument are (i) mechanically constrained to a common plane in a connected configuration, and (ii) not mechanically constrained to a common plane in a released configuration, said connected configuration being useful for maintaining visibility of the first biopsy instrument in ultrasound images derived from the linear ultrasound probe.

13. An apparatus for facilitating image-guided breast biopsy procedures, comprising:
a biopsy table for supporting a patient in a prone position, the biopsy table having an opening through which a breast of the prone patient projects;
an ultrasound-assisted biopsy platform comprising an ultrasound probe, a first biopsy guide, a first biopsy instrument, and first and second compressive surfaces, the first and second compressive surfaces for compressing the breast along a first plane generally perpendicular to said biopsy table;
an x-ray-assisted biopsy platform comprising an x-ray source, an x-ray detector, a second biopsy guide, a second biopsy instrument, and third and fourth compressive surfaces for compressing the breast along a second plane generally perpendicular to said biopsy table; and
a display system coupled to receive medical images derived from said ultrasound probe and said x-ray detector and to display said medical image information;
wherein said ultrasound-assisted biopsy platform and said x-ray-assisted biopsy platform are movably interchangeable relative to the opening such that ultrasound assisted biopsy and x-ray-assisted biopsy of the breast can be provided at different times.

14. The apparatus of claim 13, wherein said ultrasound-assisted biopsy platform and said x-ray-assisted biopsy platform are located on separate mountings each pivotably disposed around a pivot axis, whereby said movable interchange of said ultrasound-assisted biopsy platform and said x-ray-assisted biopsy relative to said opening comprises a rotational interchange achieved by rotation of said mountings around said pivot axis.

15. The apparatus of claim 13, wherein said ultrasound-assisted biopsy platform and said x-ray-assisted biopsy platform are located on a common mounting, whereby said movable interchange of said ultrasound-assisted biopsy platform and said x-ray-assisted biopsy relative to said opening comprises a translational interchange achieved by translation of said common mountings relative to said opening.

16. The apparatus of claim 13, said first compressive member comprising a first surface that contacts the breast during compression thereof and a second surface opposite said first surface, wherein said ultrasound probe is swept across said second surface to generate an ultrasound volume representative of the breast while compressed between said first and second compressive members.

17. The apparatus of claim 16, wherein said first compressive member comprises one of a vented membrane and a taut fabric sheet substantially porous to an acoustic couplant liquid or gel.

18. The apparatus of claim 17, said first biopsy needle for puncturing through a skin of the breast and capturing cellular tissue therein during a biopsy procedure, said first biopsy guide supportably coupled to said first biopsy needle for facilitating constrained linear movement of said first biopsy instrument within a third plane during said biopsy procedure, said third plane being generally perpendicular to said first plane such that said first biopsy needle also punctures through said first compressive member during said biopsy procedure.

19. An apparatus for facilitating image-guided breast biopsy procedures, comprising:
a biopsy table for supporting a patient in a prone position, the biopsy table having a first opening through which the prone patient's breast projects;
an ultrasound-assisted biopsy system comprising an ultrasound probe, a first biopsy guide, a first biopsy instrument, and first and second compressive members, the first and second compressive members for compressing the breast along a compression plane generally perpendicular to said biopsy table;
an x-ray-assisted biopsy platform comprising an x-ray source, an x-ray detector, a second biopsy guide, a second biopsy instrument, and third and fourth compressive members for compressing the breast along said compression plane;
a display system coupled to each of said ultrasound-assisted biopsy system and said x-ray-assisted biopsy system for displaying medical image information generated thereby;
wherein said first and third compressive members are substantially adjacent, jointly movable along a direction substantially parallel to said compression plane, and coplanar along respective surfaces thereof that contact a first side of the breast;
wherein said second and fourth compressive surfaces are substantially adjacent, jointly movable along the direction substantially parallel to said compression plane, and coplanar along respective surfaces thereof that contact a second side of the breast opposite the first side;
wherein said apparatus is configured to be slidably switched between an ultrasound-assisted biopsy mode and an x-ray assisted biopsy mode, the breast remaining substantially motionless as said first and third compressive members are slidably interchanged beneath said first opening and as said second and fourth compressive members are slidably interchanged beneath said first opening.

20. The apparatus of claim 19, said first compressive member comprising a first surface that contacts the first side of the breast during compression thereof and a second surface opposite said first surface, wherein said ultrasound-assisted biopsy system is configured such that said ultrasound probe is swept across said second surface to generate an ultrasound volume representative of the breast while compressed between said first and second compressive members in said ultrasound-assisted biopsy mode

21. The apparatus of claim 20, wherein said first compressive member comprises one of a vented membrane and a taut fabric sheet substantially porous to an acoustic couplant liquid or gel.

22. The apparatus of claim 20, said third compressive surface member forming a second opening through which said second biopsy instrument is inserted to puncture the first side of the breast during said x-ray assisted biopsy mode.

23. The apparatus of claim 22, said first biopsy instrument for puncturing through a skin of the breast and capturing cellular tissue therein during an ultrasound-assisted biopsy procedure, wherein said first biopsy guide is configured for facilitating constrained linear movement of said first biopsy instrument within a plane substantially parallel to said compression plane during said ultrasound-assisted biopsy procedure.
